(19) United States
(12) Patent Application Publication
(10) Pub. No.: US 2013/0041261 A1
(43) Pub. Date: Feb. 14, 2013

(54) METHOD AND SYSTEM FOR MULTI-GRID TOMOGRAPHIC INVERSION TISSUE IMAGING

(71) Applicants: Cuiping Li, Troy, MI (US); Nebojsa Duric, Bloomfield Hills, MI (US)

(72) Inventors: Cuiping Li, Troy, MI (US); Nebojsa Duric, Bloomfield Hills, MI (US)

(21) Appl. No.: 13/566,778

(22) Filed: Aug. 3, 2012

Related U.S. Application Data
(60) Provisional application No. 61/594,864, filed on Feb. 3, 2012.
(60) Provisional application No. 61/522,598, filed on Aug. 11, 2011.

(51) Int. Cl. A61B 8/15

(52) U.S. Cl. 600/442; 600/447

(57) ABSTRACT

The method of one embodiment for multi-grid tomographic inversion tissue imaging comprises receiving acoustic waveform data characterizing a volume of tissue, determining and refining models of the distributions of a first and second acoustomechanical parameter within the volume of tissue using a series of grids with progressively finer discretization levels, and generating an image based on at least one of the refined models of the first and second acoustomechanical parameters. The system of one embodiment for multi-grid tomographic inversion tissue imaging comprises ultrasound emitters configured to surround and emit acoustic waveforms toward a volume of tissue, ultrasound receivers configured to surround tissue and receive acoustic waveforms, and a processor configured to determine and refine models of the distributions of a first and second acoustomechanical parameter within a volume of tissue, and generate an image based on at least one of the refined models of the first and second acoustomechanical parameters.

### CROSS-REFERENCE TO RELATED APPLICATIONS

This application claims the benefit of U.S. Provisional Patent Application Ser. No. 61/522,598, entitled “Multi-grid Tomographic Inversion For Breast Ultrasound Sound Speed Imaging” and filed 11 Aug. 2011, and U.S. Provisional Patent Application Ser. No. 61/594,864, entitled “Multi-grid Tomographic Inversion for Breast Ultrasound Imaging” and filed 3 Feb. 2012, the entirety of which are incorporated herein by these references.

### TECHNICAL FIELD

This invention relates generally to the medical imaging field, and more specifically to a new and useful method and system for multi-grid tomographic inversion tissue imaging.

### BACKGROUND

Recent studies have demonstrated the effectiveness of ultrasound tomography imaging in detecting breast cancer. However, conventional fixed-grid methods suffer from artifacts related to over-iterated fine scale features and blurring related to under-iterated coarse scale features because fine scale features in breasts converge faster than coarse scale features. Another major barrier to the use of inverse problem techniques has been the computation cost of the conventional fixed-grid methods. These computational challenges are only made more difficult by concurrent trends toward larger data sets and correspondingly higher resolution images.

Thus, there is a need in the medical imaging field to create an improved method and system for tomographic inversion tissue imaging. This invention provides such an improved method and system for tomographic inversion tissue imaging.

### BRIEF DESCRIPTION OF THE FIGURES

FIGS. 1A-1C are schematics of the system, a perspective schematic view of a transducer ring, and a top schematic view of the transducer ring, respectively, of a preferred embodiment;

FIG. 2 is a schematic of the processor of the system of a preferred embodiment;

FIG. 3 is a flowchart depicting a method of a preferred embodiment and variations thereof;

FIG. 4 is a representation of grids used in a fixed-grid inversion technique, and for comparison, an embodiment of grids used in a multi-grid inversion technique;

FIG. 5A shows example processed images produced by a fixed grid approach;

FIG. 5B shows example processed images produced by a multi-grid approach; and

FIG. 5C shows example processed images showing the distribution of an acoustomechanical parameter within a volume of tissue produced by a multi-grid approach (left), a fixed-grid approach (middle), and x-ray computed tomography (right).

### DESCRIPTION OF THE PREFERRED EMBODIMENTS

The following description of preferred embodiments of the invention is not intended to limit the invention to these preferred embodiments, but rather to enable any person skilled in the art to make and use this invention.

### 1. System

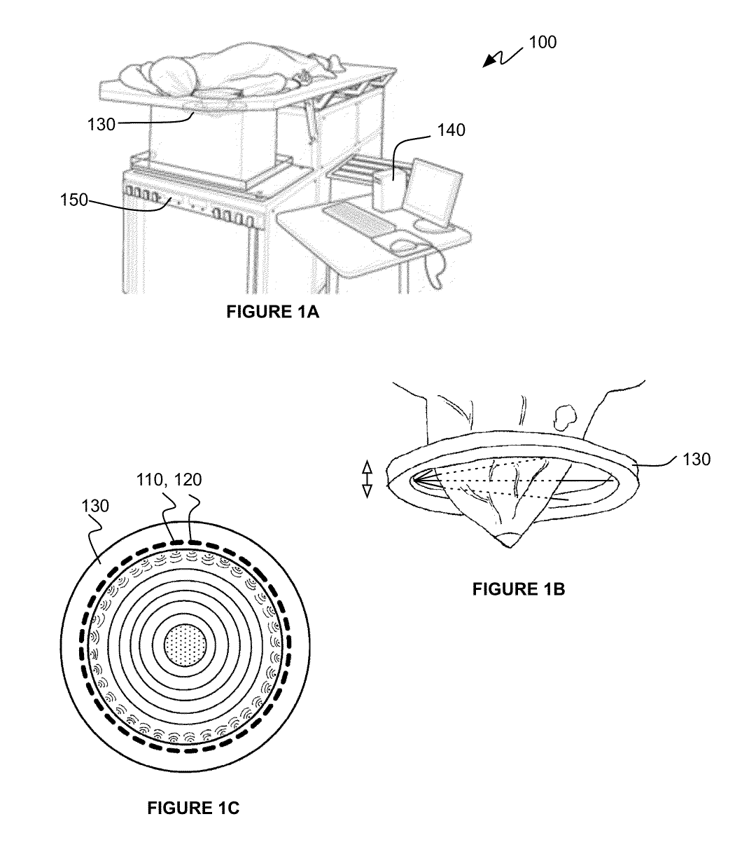

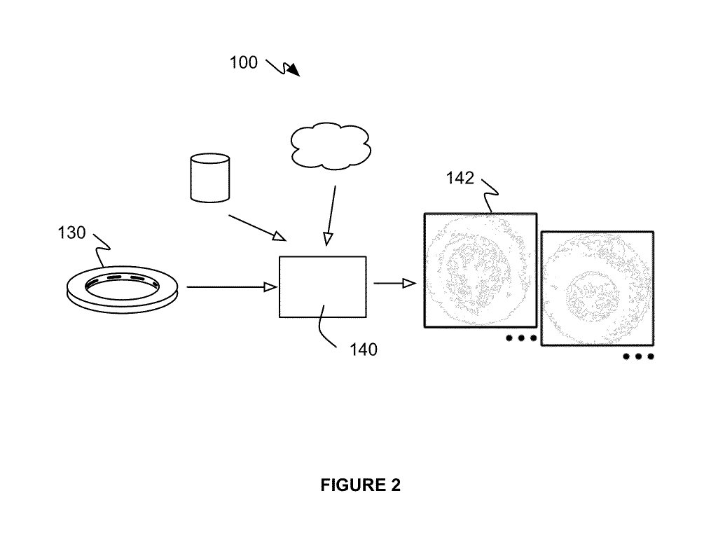

As shown in FIGS. 1-2, the system 100 of a preferred embodiment for multi-grid tomographic inversion tissue imaging comprises: an array of ultrasound emitters 110 configured to surround a volume of tissue and emit acoustic waveforms toward the volume of tissue; an array of ultrasound receivers 120 configured to surround the volume of tissue and to receive acoustic waveforms scattered by the volume of tissue; and a processor 140 configured to receive a data set representative of acoustic waveforms originating from the array of ultrasound emitters surrounding the volume of tissue, scattered by the volume of tissue, and received with the array of ultrasound receivers surrounding the volume of tissue, determine, using a first grid of a series of grids with progressively finer discretization levels, a first initial model of the distribution of a first acoustomechanical parameter within the volume of tissue, refine the first initial model using each grid in the series of grids to determine a first series of refined models, comprising a first final model, determine a second initial model of the distribution of a second acoustomechanical parameter within the volume of tissue based on the first initial model, refine the second initial model based on each refined model in the first series of refined models and using each grid in the series of grids to determine a second series of refined models, comprising a second final model, and generate an image of the volume of tissue based on at least one of the first and second final models. The processes performed by the preferred processor are described in further detail below. The system 100 is preferably used to image a volume of tissue, such as breast tissue, for screening and/or diagnosis of cancer within the volume of tissue. In other applications, the system can be used to characterize regions of interest in the tissue (e.g., to characterize suspicious masses as a tumor, a fibroadenoma, a cyst, another benign mass, or any suitable classification) or for monitoring status of the tissue such as during a cancer treatment. However, the system can be used in any suitable application for imaging any suitable kind of tissue with ultrasound tomography.

As shown in FIG. 1C, the preferred system 100 can include an array of ultrasound emitters 110 and ultrasound receivers 120. The array of ultrasound emitters no preferably functions to irradiate the volume of tissue with acoustic waveforms from multiple locations distributed around the volume of tissue. The array of ultrasound receivers 120 preferably functions to receive the acoustic waveforms, a portion of which are preferably scattered by the volume of tissue. In a preferred embodiment, as shown in FIG. 1C, the arrays of ultrasound emitters no and receivers 120 surround the tissue such that each ultrasound emitter 110 is flanked by and is adjacent to at least two other ultrasound emitters, and/or each ultrasound receiver 120 is flanked by and is adjacent to at least two other ultrasound receivers. In other words, the ultrasound emitters 110 and the ultrasound receivers 120 are preferably arranged in a substantially continuous and/or contiguous manner surrounding the tissue. By irradiating adjacent common emitter waveforms from locations collectively surrounding the tissue, the ultrasound emitters 110 provide data coverage that is more homogeneous and denser than standard ultrasound systems having linear ultrasound emitter arrays. Furthermore, by receiving adjacent common receiver waveforms, the ultrasound receivers 120 provide increased accuracy of cross-correlation of physically adjacent waveforms, thereby resulting in a higher-quality acoustic speed rendering of the volume of tissue.

In particular, the ultrasound emitters 110 and ultrasound receivers 120 are preferably arranged in an axially symmetrical arrangement. More preferably, in an exemplary embodiment shown in FIGS. 1A-1C, the system 100 includes a scanning apparatus including a ring-shaped transducer 130 that includes tissue-encircling arrays of ultrasound emitters 110 and receivers 120 for scanning breast tissue of a patient. As shown in FIG. 1A, during a scan, the patient positions herself or himself facedown on a flexible bed having a hole in a chest region of the bed. As shown in FIG. 1B, the breast tissue of the patient passes through the hole in the bed and is positioned such that the transducer 130 surrounds the tissue. The transducer 130 can preferably be immersed in a tank of water or another suitable acoustic coupling medium, and can be fixed to a gantry that moves the transducer 130 in a path to pass along the tissue in an anterior-posterior direction, thereby preferably imaging the entire breast (or alternatively a selected portion of the breast or other suitable tissue).

In one specific variation of the preferred system 100, the ring transducer 130 includes 256 evenly distributed ultrasound elements that each emits a fan beam of ultrasound signals towards the breast tissue and opposite end of the ring, and receives ultrasound signals scattered by the breast tissue (e.g., transmitted by and/or reflected by the tissue) during scanning of the tissue. In one example, the transmitted broadband ultrasound signals have a central frequency around 2 MHz and the received ultrasound signals are recorded at a sampling rate of 8.33 MHz. However, the ring transducer may have any suitable number of elements that emit and record ultrasound signals at any suitable frequencies.

As shown in FIG. 1A and FIG. 2, the preferred system 100 includes a processor 140. The processor 140 preferably functions to generate a rendering or image of the volume of tissue based on the received acoustic waveforms. In particular, the processor 140 is preferably configured to generate a rendering or image of the volume of tissue by determining initial models of the distributions of a first and second acoustomechanical parameter within a volume of tissue, wherein the initial model of the second acoustomechanical parameter is based on the initial model of the first acoustomechanical parameter, refining the initial models using a series of grids with progressively finer discretization levels, and generating an image of the volume of tissue based on at least one of the final models of the first and second acoustomechanical parameters. The processor 140 is preferably configured to perform the method further described below.

As shown in FIG. 2, the preferred processor 140 can be configured to produce one or more two-dimensional image slices 142 of the tissue under examination, based on at least one of the refined models of the first and second acoustomechanical parameters. Preferably, the acoustomechanical parameter rendering can include two-dimensional image slices 142 of the tissue corresponding to respective cross-sections of the volume of tissue (e.g., image slices of discrete anterior-posterior positions of breast tissue), and/or a three-dimensional rendering resulting from a composite of multiple two-dimensional cross-sectional images, or alternatively resulting from scanning the volume of tissue in a three-dimensional manner. In some applications, the acoustomechanical parameter rendering can be combined and/or compared with additional renderings of the volume of tissue based on other acoustomechanical parameters (e.g., attenuation, reflection).

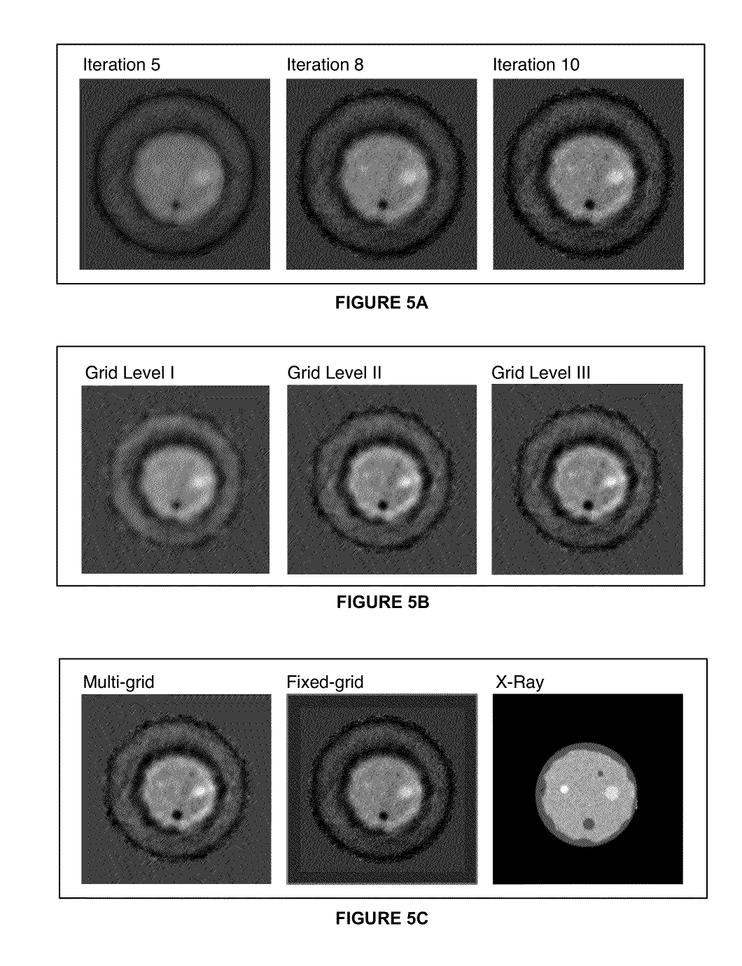

An embodiment of the system can be used to produce images based on both in vitro and in vivo ultrasound data acquired using a ring transducer 130. Examples of a cross-sectional sound speed images for a breast phantom are shown in FIG. 5C (multi-grid tomography, left) and FIG. 5C (fixed-grid tomography, middle).

As shown in FIG. 1A, the preferred system 100 can further include a controller 150 that controls the transducer 130 and its ultrasound emitters and receivers no, 120 (e.g., speed of transducer movement, activation of ultrasound emitters and/or receivers). Furthermore, in alternative embodiments, scanning may be performed with any suitable transducer having arrays of ultrasound emitters and ultrasound receivers surrounding the volume of the tissue. The processor 140 can be coupled directly to the scanning apparatus (e.g., part of a local workstation in direct communication to the ultrasound emitters and receivers), and/or can be communicatively coupled to a storage device (e.g., a server or other computer-readable storage medium) to receive data representative of the received acoustic waveforms.

### 2. Method

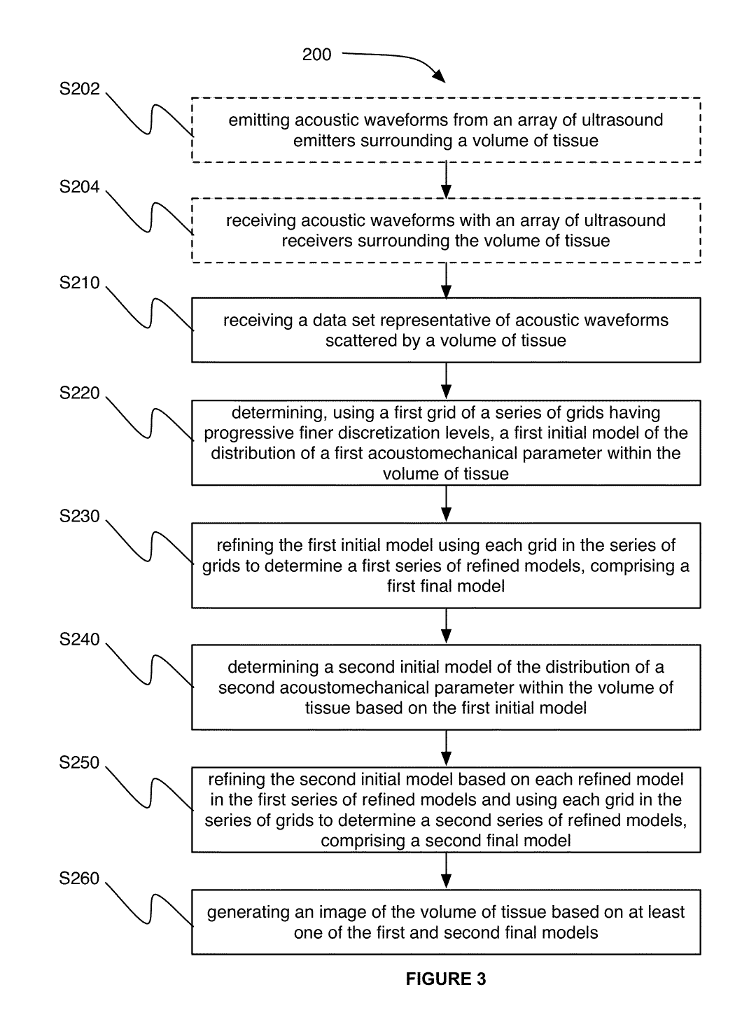

As shown in FIG. 3 the method 200 of a preferred embodiment for multi-grid tomographic inversion tissue imaging comprises: in block S210, receiving a data set representative of acoustic waveforms scattered by the volume of tissue; in block S220, determining a first initial model of the distribution of a first acoustomechanical parameter within the volume of tissue based on the received data set; in block S230, progressively refining the first initial model to determine a first series of refined models of the distribution of the first acoustomechanical parameter within the tissue; in block S240, determining a second initial model of the distribution of a second acoustomechanical parameter within the volume of tissue based on the first initial model; in block S250, progressively refining the second initial model based on each model in the first series of refined models; and in block S260 generating an image of the volume of tissue based on at least one of the refined models of the first and second acoustomechanical parameters. In particular, the preferred method may be used to image breast tissue, for screening and/or diagnosis of cancer within the tissue. In other applications, the preferred method may be used to characterize regions of interest in the tissue (e.g., to characterize suspicious masses as a tumor, a fibroadenoma, a cyst, another benign mass, or any suitable classification) or for monitoring status of the tissue such as during cancer treatment. However, the preferred method and/or any variations thereof can be used in any suitable application for imaging any suitable kind of tissue with ultrasound tomography.

As shown in FIG. 3, block S210, which recites receiving a data set representative of acoustic waveforms scattered by a volume of tissue, functions to obtain acoustic waveform data for determining models of the distribution of a first and a second acoustomechanical parameter within the volume of tissue. In a preferred embodiment of the method 200, block S210 includes receiving data directly from a transducer. In alternative variations, block S210 includes receiving data from a computer-readable medium or storage, such as a server, cloud storage, hard drive, flash memory, optical device (CD or DVD), or other suitable device capable of receiving, storing, and/or otherwise transferring data.

Block S220 recites determining, using a first grid of a series of grids having progressively finer discretization levels, a first initial model of the distribution of a first acoustomechanical parameter within the volume of tissue. Block S220 functions to generate an initial model of the distribution of the first acoustomechanical parameter at the highest grid discretization level for further refinement using the series of grids. Block S220 is preferably performed by carrying out tomographic inversion based on the received data set, where the transformation process in the inverse problem may be expressed as Ax=b, mathematically. In this equation, A is a system matrix that describes the system sensitivity, x is a model parameter vector of the inverted pixel value for the acoustomechanical parameter being modeled, and b is a vector of the measured data. In the preferred embodiment the first acoustomechanical parameter being modeled is sound speed, and the measured data is time-of-flight data. In the tomographic inversion process, forward modeling is preferably performed by determining matrix A (ray tracing a sound speed field in the preferred embodiment) using a first grid of a series of grids with progressively finer discretization levels. In the tomographic inversion process, inverse modeling comprises solving for the model parameter vector, x, using the measured data vector, b, using the first grid of a series of grids with finer discretization levels. Block S220 is preferably performed by iteratively conducting forward and inverse modeling until a measured difference between solution iterations is below a threshold, to reach a first initial model of the distribution of the first acoustomechanical parameter within the tissue. In the preferred embodiment, where sound speed is the first acoustomechanical parameter being inverted, tomographic inversion involves using a non-linear conjugate gradient (NLCG) method with a restarting strategy.

In a variation of block S220, another acoustomechanical parameter (e.g. backscatter coefficient) could be modeled, and in another variation of block S220, iteratively performing forward and inverse modeling can alternatively be performed for a set number of iterations, as opposed to iterating until a threshold is reached. Another variation of block S220 includes conducting tomographic inversion using an alternative non-linear solution method (e.g. Backus-Gilbert method).

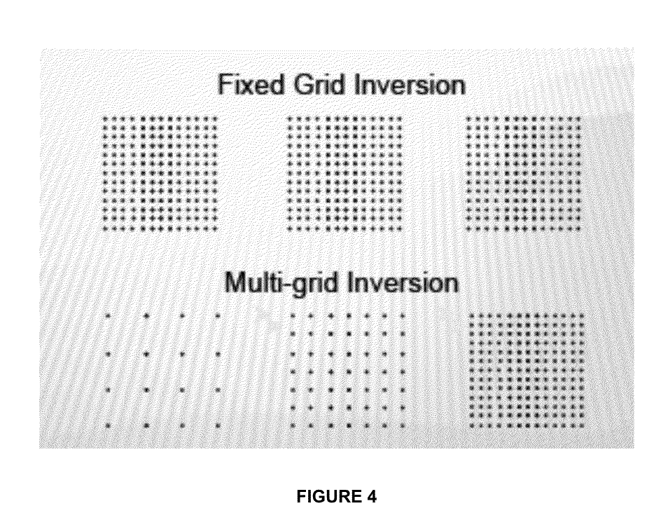

Block S230, which recites successively using each grid in the series of grids, progressively refining the first initial model to determine a first series of refined models of the distribution of the first acoustomechanical parameter within the tissue, wherein the first series of refined models comprises a first final model, functions to refine the first initial model of the first acoustomechanical parameter until fine-scale and coarse-scale features converge at the finest grid discretization level. The refined model at the finest discretization level is the first final model of the first acoustomechanical parameter being modeled, and can be used to generate an image of the distribution of the first acoustomechanical parameter within the tissue. Multi-grid tomographic inversion thus comprises the process of using multiple grids with different grid discretization levels to generate a breast ultrasound image. As shown in FIG. 4, fixed grid tomographic inversion uses a series of grids with fixed grid dimensions for forward and inverse modeling, whereas multi-grid tomographic inversion preferably uses a series of grids with finer discretization levels. As shown in FIG. 5A, a fixed-grid approach produces images with processing artifacts after iterating in the tomographic inversion process. An embodiment of the system 100 and/or method 200, produces images with fewer processing artifacts by a multi-grid approach (FIG. 5B).

In the preferred embodiment, block S230 is preferably performed by adapting the first initial model of the first acoustomechanical parameter onto the next finer grid level, using the series of grids with progressively finer grid discretization levels. Using the model adapted from the first initial model onto the next finer grid level, forward and inverse modeling are iteratively performed until convergence is reached, where convergence is preferably defined as the state where a measured difference in iterated model solutions is below a threshold value. This produces a refined model at the current grid level. Once convergence is reached at the current grid level, the refined model of the first acoustomechanical parameter at the current grid level is preferably adapted to the next finer grid level in the series of grids, and forward and inverse modeling are performed at this next finer grid level until convergence is reached. In the preferred embodiment of the method 200, the processes in block S230 of adapting a refined model of the first acoustomechanical parameter at a current grid level onto the next finer grid level, and iteratively performing forward and inverse modeling at the current grid level until convergence is reached are preferably performed until a refined model at the finest grid level in the series of grids is reached, producing a first final model of the first acoustomechanical parameter. In a variation of block S230, the processes of adapting a refined model at a current grid level onto the next finer grid level, and iteratively performing forward and inverse modeling at the current grid level until convergence is reached can alternatively be performed until a measured difference between refined solutions at a grid level and a subsequent grid level is below a threshold.

Block S240, which recites determining a second initial model of the distribution of a second acoustomechanical parameter within the volume of tissue based on the first initial model, functions to generate an initial model of the distribution of the second acoustomechanical parameter at the highest grid discretization level for further refinement using the series of grids and the first series of refined models of the distribution of the first acoustomechanical parameters within the tissue. Block S240 is preferably performed by carrying out tomographic inversion based on the first final model of the first acoustomechanical parameter, where the transformation process in the inverse problem may be expressed as Ax=b, mathematically. In this equation, A is a system matrix that describes the system sensitivity, x is a model parameter vector of the inverted pixel value for the acoustomechanical parameter being modeled, and b is a vector of the measured data. In the preferred embodiment the second acoustomechanical parameter being modeled is attenuation, and the measured data is integrated attenuation coefficient data. In the tomographic inversion process, forward modeling is preferably performed by determining matrix A using a first grid of a series of grids with progressively finer discretization levels. In the tomographic inversion process, inverse modeling comprises solving for the model parameter vector, x, using the measured data vector, b, and using the first grid of a series of grids with finer discretization levels. In the preferred embodiment, block S240 is preferably performed by iteratively conducting forward and inverse modeling until a measured difference between solution iterations is below a threshold, to reach a second initial model of the distribution of the second acoustomechanical parameter within the tissue. In the preferred embodiment, where attenuation is the second acoustomechanical parameter being modeled, tomographic inversion involves using a least squares (LSQR) method to solve the linear inverse problem.

In the preferred embodiment of the method 200, determining the second initial model based on the first initial model in block S240 comprises determining an initial model of attenuation within the tissue based on the first initial model of sound speed within the tissue. Basing attenuation on sound speed is preferably performed by using a frequency domain approach, where attenuation is parameterized using a complex-valued sound-speed parameter characterized by the expression

υ=υᵣ−i(υᵣ)/(2Q)

where υ is the complex-valued sound-speed, υᵣ is the real-valued sound speed, i is the imaginary unit, and Q is the quality factor, a dimensionless parameter related to the loss in energy. Attenuation is proportional to the inverse factor Q⁻1, and to frequency.

In a variation of block S240, solving the linear inverse problem to invert attenuation data can alternatively be performed using inversion methods for large-scale systems, comprising subspace approximation, Monte-Carlo simulation, regression, and low-dimensional vector operations. Alternatively, block S240 can be performed using iterative shrinkage-thresholding algorithms in another variation.

In another variation of block S240, the second acoustomechanical parameter can alternatively be an acoustomechanical parameter other than sound speed (e.g. reflectivity) derived from ultrasound data.

Block S250, which recites successively using each grid in the series of grids, progressively refining the second initial model based on each model in the first series of refined models to determine a second series of refined models of the distribution of a second acoustomechanical parameter within the tissue, wherein the second series of refined models comprises a second final model, functions to refine the second initial model of the second acoustomechanical parameter until fine-scale and coarse-scale features converge at the finest grid discretization level. In the preferred embodiment, the refined model at the finest discretization level is the second final model of the second acoustomechanical parameter being modeled, and can be used to generate an image of the distribution of the second acoustomechanical parameter within the tissue.

In the preferred embodiment, block S250 is preferably performed by adapting the second initial model of the second acoustomechanical parameter onto the next finer grid level, using the series of grids with progressively finer grid discretization levels. Using the model adapted from the second initial model onto the next finer grid level, forward and inverse modeling are iteratively performed until convergence is reached, where convergence is preferably defined as the state where a measured difference in iterated model solutions is below a threshold value. This produces a refined model at the current grid level. Once convergence is reached at the current grid level, the refined model at the current grid level is preferably adapted to the next finer grid level in the series of grids, and the measured data vector, b, is updated at this next finer grid level. In the preferred embodiment, b is a vector of measured data (e.g. integrated attenuation coefficients based on the received ultrasound data), and is updated at this next finer grid level during the determination of matrix A, the matrix that defines system sensitivity. As an example, the matrix A can be determined by tracing ray paths at this next finer grid level. Forward modeling and inverse modeling are performed at this next finer grid level, using the refined model of the first acoustomechanical parameter at this next finer grid level, until convergence is reached to arrive at a refined model of the second acoustomechanical parameter at this next finer grid level. The refined model at the current grid level is then adapted to the next grid with a finer discretization level in the series of grids with finer discretization levels.

In the preferred embodiment of the method 200, the processes in block S250 of adapting a refined model of the second acoustomechanical parameter at a current grid level onto the next finer grid level, and iteratively performing forward and inverse modeling (using an updated measured data vector b and the refined model of the first acoustomechanical at the current grid level) until convergence is reached are preferably performed until a refined model of the second acoustomechanical parameter at the finest grid level in the series of grids is reached, producing a second final model of the second acoustomechanical parameter.

In a variation of block S250, the processes of adapting a refined model at a current grid level onto the next finer grid level, and iteratively performing forward and inverse modeling at the current grid level until convergence is reached can alternatively be performed until a measured difference between refined solutions at a grid level and a subsequent grid level is below a threshold.

In the preferred embodiment of the method 200, a refined model of sound speed distribution is determined immediately prior to the determination of a refined model of attenuation distribution at each grid level. In an alternative embodiment of the method 200, blocks S220 and S230 can be performed prior to blocks S240 and S250, such that the first final model of the first acoustomechanical parameter is determined prior to the determination of the second initial model of the second acoustomechanical parameter. In another alternative of the method 200, a refined model of the distribution of the second acoustomechanical parameter within the tissue at a given grid level is determined after a refined model of the distribution of the first acoustomechanical parameter with the tissue at the same grid level is determined.

In an alternative embodiment of the method 200, a separate series of grids for forward and inverse modeling may be used, wherein a first series of grids, comprising a number of grids with progressively finer discretization levels, is used for forward modeling, and a second series of grids, comprising a number of grids with progressively finer discretization levels, is used for inverse modeling. In this alternative, the discretization levels of the first and second series of grids may or may not be substantially the same. In another alternative embodiment of the method 200, each grid used for forward and/or inverse modeling may or may not have uniform grid dimensions; in this embodiment, the average grid dimension at each grid discretization level is less fine than the average grid dimension at the next finer grid discretization level in the series of grids.

In the preferred embodiment, adapting a refined model at the current grid level onto the next finer grid level is preferably performed by interpolating the refined model from the final iteration at a grid level onto the next finer grid level in the series of grids with progressively finer discretization levels, wherein interpolation occurs between acoustomechanical parameter values determined at the nodes of a grid. In an alternate embodiment, adapting a refined model at the current grid level onto the next finer grid level is alternatively performed by using averaging to adapt the refined model from the final iteration at a grid level onto the next finer grid level in the series of grids with progressively finer discretization levels, wherein averaging involves taking a mean of acoustomechanical parameter values determined at nodes of a grid.

Block S260, which recites generating an image of the volume of tissue based on at least one of the first and second final models, functions to create a visual representation of the distribution of at least one of the first and second acoustomechanical parameters within the tissue. Preferably, block S260 comprises producing one or more two-dimensional image slices of the tissue under examination, based on at least one of the refined models of the first and second acoustomechanical parameters. Preferably, the acoustomechanical parameter rendering can include two-dimensional image slices of the tissue corresponding to respective cross-sections of the volume of tissue (e.g., image slices of discrete anterior-posterior positions of breast tissue), and/or a three-dimensional rendering resulting from a composite of multiple two-dimensional cross-sectional images. In some applications, the acoustomechanical parameter rendering can be combined and/or compared with additional renderings of the volume of tissue based on other acoustomechanical parameters (e.g., attenuation, reflection).

In the example shown in FIG. 5C, a model of the distribution of an acoustomechanical parameter refined at the finest grid level in a multi-grid approach can be used to produce an image of the distribution of an acoustomechanical parameter within a two-dimensional slice of tissue. In the example of FIG. 5C, the image produced by a multi-grid approach (FIG. 5C, left) is consistent with that produced by an x-ray computed tomography scan (FIG. 5C, right), without processing artifacts resulting from over-iteration of fine scale features captured in ultrasound data (FIG. 5C, middle).

In the preferred embodiment of the method 200, an image of the volume of tissue based on at least one of the first and second final models is generated. In a variation of the method 200, the method 200 further comprises generating an image based on one of the refined models in at least of the first series of refined models and the second series of refined models.

As shown in FIG. 3, in alternate embodiments the preferred method can further include block S202 and block S204. Block S202 recites emitting acoustic waveforms from an array of ultrasound emitters surrounding the volume of tissue, and block S204 recites receiving acoustic waveforms with an array of ultrasound receivers surrounding the volume of tissue. Blocks S202 and S204 preferably function to scan and gather ultrasound data regarding the volume of tissue. Block S202 is preferably performed with an array of ultrasound emitters in which each ultrasound emitter is flanked by, and more preferably contiguous and/or continually disposed with, at least two other ultrasound emitters and/or receivers in a circular ring or other suitable axially symmetrical transducer configured to receive and surround the volume of tissue. Similarly, block S204 is preferably performed with an array of ultrasound receivers in which each ultrasound receiver is flanked by, and more preferably contiguous with, at least two other ultrasound receivers and/or emitters in a circular ring or other suitable axially symmetrical transducer configured to receive and surround the volume of tissue. Blocks S202 and S204 are preferably performed with a ring transducer in a system as described above, but may alternatively be performed with any suitable transducer. The method may further include recording data representative of the received acoustic waveforms, such as by storing acquired imagining data in a computer readable storage medium.

The system and method of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processor 140 and/or the controller 150. The computer-readable medium can be stored on any suitable computer-readable media such as RAMs, ROMs, flash memory, EEPROMs, optical devices (CD or DVD), hard drives, floppy drives, or any suitable device. The computer-executable component is preferably a general or application specific processor, but any suitable dedicated hardware or hardware/firmware combination device can alternatively or additionally execute the instructions.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

We claim:

1. A method for multi-grid tomographic inversion tissue imaging using an array of ultrasound emitters configured to surround the volume of tissue and emit acoustic waveforms toward the volume of tissue and an array of ultrasound receivers configured to surround the volume of tissue and receive acoustic waveforms scattered by the volume of tissue, comprising:
receiving a data set representative of acoustic waveforms scattered by the volume of tissue;
determining, using a first grid of a series of grids having progressively finer discretization levels, a first initial model of the distribution of a first acoustomechanical parameter within the volume of tissue based on the received data set;
successively using each grid in the series of grids, progressively refining the first initial model to determine a first series of refined models of the distribution of the first acoustomechanical parameter within the tissue, wherein the first series of refined models comprises a first final model;
determining a second initial model of the distribution of a second acoustomechanical parameter within the volume of tissue based on the first initial model;
successively using each grid in the series of grids, progressively refining the second initial model based on each model in the first series of refined models to determine a second series of refined models of the distribution of the second acoustomechanical parameter within the tissue, wherein the second series of refined models comprises a second final model; and
generating an image of the volume of tissue based on at least one of the first and second final models.

2. The method of claim 1, further comprising generating an image based on one of the refined models in at least one of the first series of refined models and the second series of refined models.

3. The method of claim 1, wherein at least one of determining the first initial model, determining the second initial model, progressively refining the first initial model, and progressively refining the second initial model comprises iteratively solving an inverse problem at each discretization level.

4. The method of claim 3, wherein solving the inverse problem comprises iteratively performing forward and inverse modeling.

5. The method of claim 4, wherein forward modeling comprises tracing the ray paths on the grid in the series of grids.

6. The method of claim 4, wherein inverse modeling comprises performing a non-linear conjugate gradient method with restarting strategy.

7. The method of claim 4, wherein inverse modeling comprises performing a least squares method.

8. The method of claim 1, wherein progressively refining the first initial model of the first acoustomechanical parameter comprises adapting the refined model having a given discretization level to a grid having a finer discretization level in the series of grids.

9. The method of claim 8, wherein adapting the refined model having a given discretization level to a grid having a finer discretization level comprises interpolating between values determined at nodes of the grid.

10. The method of claim 8, wherein adapting the refined model having a given discretization level to a grid having a finer discretization level comprises averaging values determined at nodes of the grid.

11. The method of claim 1, wherein progressively refining the second initial model of the second acoustomechanical parameter comprises adapting the refined model having a given discretization level to a grid having a finer discretization level in the series of grids.

12. The method of claim 1, wherein the grid dimensions of each grid in the series are uniform.

13. The method of claim 1, wherein the first acoustomechanical parameter is sound speed.

14. The method of claim 1, wherein the second acoustomechanical parameter is sound attenuation.

15. The method of claim 1, wherein the first acoustomechanical parameter is sound speed, and wherein the second acoustomechanical parameter is sound attenuation.

16. The method of claim 1, wherein refining at least one model in the second series of refined models occurs before refining the first final model.

17. A system for multi-grid tomographic inversion tissue imaging comprising:
an array of ultrasound emitters configured to surround the volume of tissue and emit acoustic waveforms toward the volume of tissue;
an array of ultrasound receivers configured to surround the volume of tissue and receive acoustic waveforms scattered by the volume of tissue; and
a processer configured to:
receive a data set representative of acoustic waveforms originating from the array of ultrasound emitters surrounding the volume of tissue, scattered by the volume of tissue, and received with the array of ultrasound receivers surrounding the volume of tissue,
determine, using a first grid of a series of grids with progressively finer discretization levels, a first initial model of the distribution of a first acoustomechanical parameter within the volume of tissue based on the received data set,
successively use each grid in the series of grids to progressively refine the first initial model, thereby determining a first series of refined models of the distribution of the first acoustomechanical parameter within the tissue, wherein the first series of refined models comprises a first final model,
determine a second initial model of the distribution of a second acoustomechanical parameter within the volume of tissue based on the first initial model,
successively use each grid in the series of grids to progressively refine the second initial model based on each model in the first series of refined models to determine a second series of refined models of the distribution of the second acoustomechanical parameter within the tissue, wherein the second series of refined models comprises a second final model, and
generate an image of the volume of tissue based on at least one of the first and second final models.

18. The system of claim 17, further comprising a ring transducer that houses the array of ultrasound emitters and array of ultrasound receivers.

19. The system of claim 17, wherein the processor further generates an image based on a refined model in at least one of the first series of refined models and the second series of refined models.

20. The system of claim 17, wherein in performing at least one of determining the first initial model, determining the second initial model, progressively refining the first initial model, and progressively refining the second initial model, the processor iteratively solves an inverse problem at each discretization level.

21. The system of claim 20, wherein in solving the inverse problem, the processor iteratively performs forward and inverse modeling.

22. The system of claim 21, wherein in performing forward modeling, the processor traces the ray paths on the grid in the series of grids.

23. The system of claim 17, wherein in progressively refining the initial model of the first acoustomechanical parameter, the processor adapts the model determined at a given discretization level to a grid at a finer discretization level in the series of grids.

24. The system of claim 23, wherein in adapting the model determined at a given discretization level to a grid having a finer discretization level, the processor interpolates between values determined at nodes of the grid.

25. The system of claim 17, wherein in progressively refining the second initial model of the second acoustomechanical parameter, the processor adapts the refined model having a given discretization level to a grid having a finer discretization level in the series of grids.

26. A method for multi-grid tomographic inversion tissue imaging, comprising:
receiving a data set representative of acoustic waveforms originating from an array of ultrasound emitters surrounding the volume of tissue, scattered by the volume of tissue, and received with an array of ultrasound receivers surrounding the volume of tissue;
determining, using a first grid of a series of grids with progressively finer discretization levels, an initial model of the distribution of an acoustomechanical parameter within the volume of tissue based on the received data set;
successively using each grid in the series of grids, progressively refining the initial model to determine a series of refined models of the distribution of the acoustomechanical parameter within the tissue, wherein the series of refined models comprises a final model;
generating an image of the volume of tissue based on the final model.
